(19) United States
(12) Patent Application Publication
(10) Pub. No.: US 2010/0035911 A1
(43) Pub. Date: Feb. 11, 2010

(54) DRUG COMBINATIONS TO TREAT HYPERPROLIFERATIVE DISORDERS

(76) Inventors: Barry James Maurer, Idalou, TX (US); C. Patrick Reynolds, Lubbock, TX (US)

Correspondence Address: MYERS BIGEL SIBLEY & SAJOVEC, PO BOX 37428, RALEIGH, NC, 27627, US

(21) Appl. No.: 12/301,089

(22) PCT Filed: May 16, 2007
(86) PCT No.: PCT/US07/11686

§ 371 Date: Mar. 27, 2009

Related U.S. Application Data
(60) Provisional application No. 60/800,954, filed on May 17, 2006.

(51) Int. Cl. A61K 31/167; A61K 31/4745; A61K 31/337; A61P 35/00; A61K 31/44

(52) U.S. Cl. 514/283; 514/613; 514/449; 514/353

(57) ABSTRACT

A method of treating a hyperproliferative disorder, including a cancer, in a subject in need of such treatment, comprising administering to said subject a pharmaceutical combination containing a treatment effective amount of: (a) a vitamin A derivative (i.e., a retinoid), or a pharmaceutically acceptable salt thereof, and an inhibitor of microtubule structure or function; or (b) a combination containing fenretinide (i.e., N-(4-hydrophenyl) retinamide, 4-HPR) and ABT-751 (i.e., N-[2-[(4-hydroxyphenyl)amino]-3-pyridinyl]-4-methoxybenzenesulfonamide). Vitamin A derivatives that may be useful for this invention according to (a) include, but are not limited to, all-trans-retinoic acid, 13-cis-retinoic acid, and fenretinide. Microtubule inhibitors that may be useful for this invention according to (a) include, but are not limited to, inhibitors of the Vinca binding domain (e.g., vincristine, vinblastine, vinorelbine, and cryptophycin 52), inhibitors of the Taxane domain (e.g., paclitaxel, docetaxel, and epothilones), and inhibitors of the colchicine site (e.g., colchicine, ABT-751, CI-980, and combretastatin). A preferred retinoid according to (a) is fenretinide. A preferred microtubule inhibitor according to (b) is ABT-751.

### RELATED APPLICATIONS

This application claims priority from U.S. Provisional Application Ser. No. 60/800,954, filed May 17, 2006, the disclosure of which is incorporated by reference herein in its entirety.

### FIELD OF THE INVENTION

The present invention concerns useful drug combinations for the treatment of hyperproliferative disorders, including cancers.

### BACKGROUND OF THE INVENTION

Fenretinide [HPR; all-trans-N-(4-hydroxyphenyl)retinamide; CAS Registry number 65646-68-6] is currently believed to effect cytotoxicity in cancer cells by mechanisms that include generating reactive oxygen species and by altering sphingolipid metabolism. See, e.g. D. Delia et al., Carcinogenesis 18, 943-948 (1997); N. Oridate et al., J. Nat. Cancer Inst. 89, 1191-1198 (1997); B. Maurer, et al., J. Natl. Cancer Inst. 92, 1897-1909 (2000).

Fenretinide [HPR; all-trans-N-(4-hydroxyphenyl)retinamide; CAS Registry number 65646-68-6] is a synthetic retinoic acid derivative having the structure:

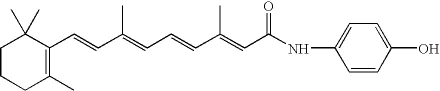

Fenretinide is minimally soluble in aqueous solution. U.S. Pat. No. 4,665,098 by Gibbs describes an oral pharmaceutical capsule composition of fenretinide as useful for the treatment of breast and bladder cancer. However, there has been limited clinical evidence for anticancer activity for fenretinide using this composition as tested in multiple Phase 1, 2, and 3 clinical trials in a number of different cancer disease states. The bioavailability of this oral capsule fenretinide composition is limited and it is speculated that greater anticancer effects might be obtained if fenretinide could be delivered to achieve higher drug plasma levels. New intravenous (U.S. Pat. No. 7,169,819) and oral fenretinide formulations (U.S. Patent Appl. US2005/010621641) with increased bioavailability are currently in early stage clinical trials.

Microtubules are protein fibrils that play a central role in cellular transport, structural integrity and cellular architecture. Microtubules typically comprise 13 protofilaments, which form the wall of a tube. Each of the protofilaments consists of a head-to-tail arrangement of α/β tubulin heterodimers. Microtubules are essential for a number of cellular processes that include the transport of intracellular cargo or organelles across long distances and the assembly of the mitotic spindle. Drugs and small molecules are known that interact with microtubules to disrupt microtubule dynamics, often by either stabilizing or destabilizing the polymerized state. Some drugs, such as Podophyllotaxin, etoposide, vinblastine, vincristine and vinorelbine inhibit or disrupt microtubules and microtubule assembly. Docetaxel (or taxotere), a derivative of the natural product paclitaxel (Taxol®), is a stabilizer of tubulin interaction. In addition to the aforementioned known drugs, there are other drug destabilizers of microtubules such as cytochalasin A and E, TN-16, myoseverin, nocodazole, vindesine, the depsipetide Phomopsin A, and d-24851.

Surprisingly, despite experiments using fenretinide since the 1960's, and unlike with other chemotherapeutic drugs, there has been a paucity of literature reports on the results of combining fenretinide with inhibitors of microtubule structure, stability or function (“microtubule inhibitors”), despite these inhibitors being a common and important class of anticancer agents. A possible reason for this is that there have been no data on the mechanisms of action of retinoids and microtubule inhibitors that would suggest synergism would be likely when combining these two classes of drugs that differ a great deal in their mechanisms and effects. It is also possible that other laboratories, like ours, conducted in vitro assays of fenretinide in combination with microtubule inhibitors which failed to demonstrate cytotoxic synergy, i.e., greater than additive cancer cell killing, within their range of sensitivity. Indeed, in a rare instance of a study reporting the results combining fenretinide plus the microtubule-stabilizing agent, paclitaxel, of two lung cancer cell lines tested, one cell line demonstrated antagonism (decreased) anticancer activity over much of the dose range tested, and the second cell line demonstrated only a weak positive effect over a portion of the tested dose range, See Kalemkerian, et al, Cancer Chemother Pharmacol 43, 145-150, (1999), FIG. 2. Similar to Kalemkerian, we initially tested fenretinide together with microtubule inhibitors in vitro and were not able to demonstrate any greater killing from the combination than was observed with either drug alone (FIG. 4). Literature reports of investigations of fenretinide in combination with microtubule inhibitors against human cancer xenografts in immunocompromised mouse models are also lacking.

Clinically, fenretinide has only been combined with a microtubule-stabilizing agent, paclitaxel, in a single Phase I study of 14 patients in the context of fenretinide oral capsules obtaining low fenretinide plasma levels combined with cisplatin plus paclitaxel in refractory solid tumors See Otterson, G. A., et al, Investigational New Drugs, 23, 555-562, (2005). Severe cumulative toxicities resulted that were consistent with combined cisplatin plus paclitaxel toxicities. It was concluded that the large number of fenretinide oral. capsules that needed to be consumed limited the applicability of the regimen. The study was insufficiently powered to detect a positive interactive effect of fenretinide on the anticancer activity of the cisplatin plus paclitaxel regimen.

### SUMMARY OF THE INVENTION

Despite this paucity of published data, and our own inability to demonstrate a synergistic interaction between the anticancer activities of fenretinide and the various classes of microtubule inhibitors in human cancer cell lines in vitro, we nonetheless undertook human tumor xenograft model experiments of fenretinide plus representatives of various classes of microtubule inhibitors owing to our access to an improved oral fenretinide formulation for clinical use and our expectations of non-overlapping systemic toxicities. Unexpectedly, we observed strong positive anticancer activity interactions between fenretinide and multiple representative microtubule inhibitors. We observed tumor regression, increased survival times, and/or suppression of tumor growth in multiple different tumor xenograft models from a disparate variety of human cancers (leukemia/lymphoma, ovarian cancer, neuroblastoma) using fenretinide plus a Vinca alkaloid-class inhibitor (vincristine), a Colchicine domain-class inhibitor (ABT-751), and a taxane domain-class inhibitor (paclitaxel). These observations represent a new paradigm for the use of semi-synthetic retinoids, such as fenretinide, in the treatment of cancers and other hyperproliferative disorders, that of retinoids in combination with inhibitors of microtubule structure, stability, or function.

The present invention is based, at least in part, on the unexpected discovery that a retinoid, (fenretinide), in combination with an inhibitor of microtubule structure or function (such as vincristine, ABT-751, or paclitaxel), greatly increases the anticancer activity of these individual agents in human cancer cell lines grown as xenograft tumors in immunocompromised mice. Thus, the activity of fenretinide and other such retinoic acid derivatives against hyperproliferative disorders as defined below can be enhanced by also administering an agent that disrupts or alters cellular microtubule structure, stability, or function. Conversely, inhibitors of microtubule structure, stability, or function include but are not limited to compounds that inhibit microtubule growth, modulate the dynamics of microtubules, induce the self-association of tubulin dimers into single-walled rings and spirals, promote microtubule polymerization and/or stabilization, or induce the dissociation or depolymerization of microtubules. Such agents include but are not limited to microtubule inhibitors that function via the Vinca tubulin binding domain (e.g., vincristine, vinblastine, vinorelbine, and cryptophycin 52, inhibitors functioning via the Taxane tubulin binding domain (e.g., paclitaxel, docetaxel, and epothilones), and inhibitors functioning via the Colchicine tubulin binding domain (e.g., colchicine, ABT-751, CI-980, and combretastatin). Specific examples are given below. In the preferred embodiment, the retinoic acid derivative is given in an amount that is effective in producing anticancer activity, and the inhibitor of microtubule structure, stability, or function is given in an amount effective to increase the anticancer activity over that which would be produced by the retinoic acid derivative alone. However, as shown for some xenografted human tumors we have tested, in some instances the retinoic acid derivative alone, or the microtubule inhibitor alone, will not have substantial anticancer activity, while the two drugs in combination will have significant anticancer activity. In certain cases, the increased anticancer activity is also greater than that expected to be produced by the sum of the anticancer activity produced by the retinoic acid derivative and the inhibitor of microtubule structure, stability, or function when given separately. Anticancer activity is considered in this context to be killing cancer cells, reduction of the size of tumors, or slowing the growth of tumors or the expansion of tumor cells in blood or bone marrow.

A method of treating a hyperproliferative disorder in a subject in need of such treatment comprises or consists essentially of administering to the subject, in combination, a treatment effective amount of: (a) a retinoic acid derivative such as fenretinide or a pharmaceutically acceptable salt thereof; and (b) a microtubule inhibitor functioning via the Vinca tubulin binding domain (including the pharmaceutically acceptable salts thereof) such as vincristine or a pharmaceutically acceptable salt thereof The microtubule inhibitor functioning via the Vinca domain is administered in an amount effective to enhance the anticancer activity of the retinoic acid derivative, such that the two compounds together have an efficacious activity. Preferably, the retinoic acid derivative is given in an amount effective to produce an anticancer activity, and the microtubule inhibitor functioning via the Vinca domain is given in an amount effective to increase the anticancer activity over that which would be produced by the retinoic acid derivative alone. In certain cases, the microtubule inhibitor functioning via the Vinca domain is given in an amount necessary to effect an increase in anticancer activity that is greater than the sum of the anticancer activity expected to be produced by the retinoic acid derivative and the microtubule inhibitor functioning via the Vinca domain when given separately. Other compounds including the compounds described herein may also be administered.

Also disclosed is a method of treating a hyperproliferative disorder in a subject in need of such treatment comprises or consists essentially of administering to the subject, in combination, a treatment effective amount of: (a) a retinoic acid derivative such as fenretinide or a pharmaceutically acceptable salt thereof; and (b) a microtubule inhibitor functioning via the Taxane tubulin binding domain (including the pharmaceutically acceptable salts thereof) such as paclitaxel or a pharmaceutically acceptable salt thereof. The microtubule inhibitor functioning via the Taxane domain is administered in an amount effective to enhance the anticancer activity of the retinoic acid derivative, such that the two compounds together have an efficacious activity. Preferably, the retinoic acid derivative is given in an amount effective to produce anticancer activity, and the microtubule inhibitor functioning via the Taxane domain is given in an amount effective to increase the anticancer activity over that which would be produced by the retinoic acid derivative alone. In certain cases, the microtubule inhibitor functioning via the Taxane domain is given in an amount necessary to effect an increase in the anticancer activity that is greater than the sum of the anticancer activity expected to be produced by the retinoic acid derivative and the microtubule inhibitor functioning via the Taxane domain when given separately. Other compounds including the compounds described herein may also be administered.

Also disclosed is a method of treating a hyperproliferative disorder in a subject in need of such treatment comprises or consists essentially of administering to the subject, in combination, a treatment effective amount of: (a) a retinoic acid derivative such as fenretinide or a pharmaceutically acceptable salt thereof, and (b) a microtubule inhibitor functioning via the Colchicine tubulin binding domain (including the pharmaceutically acceptable salts thereof) such as ABT-751 or a pharmaceutically acceptable salt thereof. The microtubule inhibitor functioning via the Colchicine domain is administered in an amount effective to enhance the anticancer activity of the retinoic acid derivative, such that the two compounds together have an efficacious activity. Preferably, the retinoic acid derivative is given in an amount effective to produce anticancer activity, and the microtubule inhibitor functioning via the Colchicine domain is given in an amount effective to increase the anticancer activity over that which would be produced by the retinoic acid derivative alone. In certain cases, the microtubule inhibitor functioning via the Colchicine domain is given in an amount sufficient to effect an increase in anticancer activity that is greater than the sum of the anticancer activity expected to be produced by the retinoic acid derivative and the microtubule inhibitor functioning via the Colchicine domain when given separately. Other compounds including the compounds described herein may also be administered.

It is understood from the above description that not all combinations of retinoids plus microtubule inhibitors may be equally active in any given specific hyperproliferative disorder, that certain drug combinations are preferred in certain hyperproliferative disorders as exampled below.

It is understood from the above descriptions that more than one inhibitor of microtubule structure, stability, or function could be combined with more than one retinoid to produce the desired effect and that such drug combinations are an aspect of the instant invention. Similarly, one or more retinoids could be combined with multiple microtubule inhibitors.

It is understood in the above descriptions that an anticancer activity could be an activity that induces cell death in a cancer cell or an activity that slows the proliferation or growth of a cancer cell or of a cancer cell mass (tumor) or an activity that prolongs survival of a host with a cancer.

It is understood in the above description that a host with a cancer could be a human being or a nonhuman animal.

It is also understood from the above descriptions that said anticancer activity in a cancer disorder could also be an antiproliferative activity in a hyperproliferative disorder and that this is also an aspect of the present invention.

It is also understood that in the above descriptions that combining of a retinoic acid derivative with a microtubule inhibitor, or a combination of those drugs, refers to use of the drugs together for therapy, whether given simultaneously, or sequentially, in any order.

Formulations comprising the aforesaid combinations of compounds in a single pharmaceutical carrier or vehicle, for carrying out the foregoing treatments, are also an aspect of the instant invention.

The use of the foregoing compounds for the preparation of a medicament for carrying out the aforesaid treatments are also an aspect of the present invention.

The foregoing and other objects and aspects of the present invention are explained in detail in the drawings herein and the specification set forth below.

### BRIEF DESCRIPTION OF THE DRAWINGS

FIG. 1 Photomicrographs showing increased apoptotic cell death by TUNEL (detection of internucelosomal DNA breaks using terminal nucleotidyl transferase) assay when the multidrug resistant human neuroblastoma cell line CHLA-136 was grown as subcutaneous tumor xenografts in immunocompromised murine hosts and were treated with fenretinide+ABT-751 compared to fenretinide or ABT-751 alone.

FIG. 2 A demonstrates that the event-free survival of immunocompromised mice bearing 4 different human neuroblastoma cell lines (CHLA-90, CHLA-136, SMS-KCNR, and CHLA-140) grown as subcutaneous tumor xenografts was increased by fenretinide+ABT-751 compared to either drug alone even in CHLA-136, a xenograft that was minimally response to fenretinide or ABT-751 as single agents at the drug doses employed.

FIG. 2 B shows the tumor volume as measured with calipers from the subcutaneous tumors from mice used to derive the event-free survival log-rank analysis shown in FIG. 2 A.

FIG. 3 demonstrates that survival of immunocomprised mice bearing the human Ramos Burkitts lymphoma cell line grown as subcutaneous tumor xenografts was increased by fenretinide+vincristine compared to either drug separately even in a tumor cell line minimally responsive to fenretinide or vincristine as single agents at the drug doses employed.

FIG. 4 shows data representative of our initial in vitro testing of the microtubule inhibitor ABT-751 in combination with that the in vitro cytotoxicity of the human neuroblastoma cell lines, was not different for fenretinide+ABT-751 compared to either drug alone. Cytotoxicity was determined by DIMSCAN assay (Frgala T, Kalous O, Proffitt R T, Reynolds C P, A fluorescence microplate cytotoxicity assay with a 4-log dynamic range that identifies synergistic drug combinations, Molecular Cancer Therapeutics 2007 March; 6(3):886-97) at 4 days of exposure to various concentrations of the drugs as single agents, or together in a fixed ratio of concentrations. These were the initial data from testing of the combinations. In spite of these data, which did not point toward a synergistic interaction between microtubule inhibitors and fenretinide, we conducted in vivo experiments that obtained the surprising results shown in FIGS. 1 and 2.

FIG. 5 demonstrates that the in vitro cytotoxicity of fenretinide+ABT-751 for 2 human neuroblastoma cell lines (CHLA-140 and CHLA-119) that were identified after screening a large panel of multi-drug resistant neuroblastoma cell lines. Of all the lines tested to date, only these 2 lines show any increases in cytotoxicity for the combination relative to the single drugs in vitro. Note that unlike the more striking effect seen for all neuroblastoma cell lines tested as xenografts, even in these selected lines, only a modest increase in activity is seen with the combination relative to the single agents. Cytotoxicity was determined by DIMSCAN assay at 4 days of exposure to various concentrations of the drugs as single agents, or together in a fixed ratio of concentrations.

FIG. 6 demonstrates that survival of immunocomprised mice bearing human ovarian cancer cell line, CL-1572, grown as subcutaneous tumor xenografts is prolonged by fenretinide+paclitaxol compared to either drug separately even in a tumor cell line minimally responsive to fenretinide or paclitaxel as single agents at the drug doses employed

FIG. 7 illustrates that the combination of ABT-751+fenretinide is well tolerated in mice as demonstrated by the lack of weight loss from the mice.

### DETAILED DESCRIPTION OF THE PREFERRED EMBODIMENTS

The methods of the present invention utilize the combined effects of retinoic acid derivatives and agents (i.e., a potentiating agent) that inhibit the structure, stability or function of microtubules, in order to inhibit the growth of tumors, cancers, neoplastic tissue and other premalignant and noneoplastic hyperproliferative disorders, all of which are together referred to as hyperproliferative or hyperplastic disorders herein.

Examples of tumors, cancers and neoplastic tissue that can be treated by the present invention include but are not limited to malignant disorders such as lymphomas; ovarian cancers; breast cancers; osteosarcomas; angiosarcomas; fibrosarcomas and other sarcomas; leukemias; sinus tumors; uretal, bladder, prostate and other genitourinary cancers; colon esophageal and stomach cancers and other gastrointestinal cancers; lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas.

Examples of premalignant and nonneoplastic hyperproliferative disorders include but are not limited to myelodysplastic disorders; cervical carcinoma-in-situ; familial intestinal polyposes such as Gardner syndrome; oral leukoplakias; histiocytoses; keloids; hemangiomas; hyperproliferative arterial stenosis; EBV-induced lymphoproliferative disease, hyperkeratoses and papulosquamous eruptions including arthritis, autoimmune disorders such as lupus, inflammatory arthritis, graft-vs-host disease. The methods of treatment disclosed herein may be employed with any subject known or suspected of carrying or at risk of developing a hyperproliferative disorder as defined herein.

As used herein, “treatment” of a hyperproliferative disorder refers to methods of killing, inhibiting or slowing the growth or increase in size of a body or population of hyperproliferative cells or tumor or cancerous growth, reducing hyperproliferative cell numbers, or preventing spread to other anatomic sites, as well as reducing the size of a hyperproliferative growth or numbers of hyperproliferative cells. As used herein, “treatment” is not necessarily meant to imply cure or complete abolition of hyperproliferative growths. As used herein, a treatment effective amount is an amount effective to result in the killing, the slowing of the rate of growth of hyperproliferative cells, the decrease in size of a body of hyperproliferative cells, and/or the reduction in number of hyperproliferative cells. The potentiating agent (or agents) is included in an amount sufficient to enhance the activity of the first compound, such that the two (or more) compounds together have greater therapeutic efficacy than the individual compounds given alone (e.g., due to synergistic interaction; reduced combined toxicity, etc.).

As used herein, the administration of two or more compounds “in combination” means that the two compounds are administered closely enough in time that the presence of one alters the biological effects of the other. The two compounds may be administered simultaneously (concurrently) or sequentially. Simultaneous administration may be carried out by mixing the compounds prior to administration, or by administering the compounds at the same point in time but at different anatomic sites or using different routes of administration.

The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

Subjects to be treated by the methods of the present invention include both human subjects and animal subjects for veterinary purposes. Animal subjects are preferably mammalian subjects including horses, cows, dogs, cats, rabbits, sheep, and the like.

A variety of intracellular molecules are known to trigger or inhibit cell death (S. Rowan and D. Fisher, Leukemia 11, 457 (1997); K. Saini and N. Walker, Mol. Cell Biochem. 178, 9 (1998)). Most current work focuses on elucidating pathways for programmed cell death (apoptosis), in which triggers of apoptosis (such as DNA damage) can activate various pathways (e.g. p53, Fas, and others), which can be modulated by yet other molecules (such as the Bcl-2 family of pro- and anti-apoptotic proteins), with caspase activation being a late step in the final events leading to apoptotic cell death. However, not all cell death occurs via apoptosis, and cell death induced by fenretinide involves both apoptosis and necrosis (J. Clifford et al., Cancer Res. 59, 14 (1999); B. Maurer, et al, J. Natl Cancer Inst 91, 1138, 1999). One possible mechanism for the synergistic interaction between microtubule inhibitors and fenretinide would be via mitotic catastrophe (Castedo M, Perfettini J L, Roumier T, Andreau K, Medema R, Kroemer G., Cell death by mitotic catastrophe: a molecular definition, Oncogene. 2004 Apr. 12; 23(16):2825-37). Thus, combining 4-HPR (fenretinide) with microtubule inhibitors offers a means for enhancing the cytotoxic efficacy of 4-HPR (fenretinide) and other retinoids.

Compounds that may be used to carry out the present invention, and formulations thereof and the manner of administering the same, are described in detail below.

### 1. Retinoic Acid Derivative Active Compounds.

Retinoic acid derivatives that can be used to carry out the present invention are those generating ceramide and may include those described in U.S. Pat. No. 4,190,594 to Gander (the disclosures of all patent references cited herein are incorporated herein by reference). These retinoic acid derivatives include:

(A) esters of all-trans-retinoic acid having the following formula:

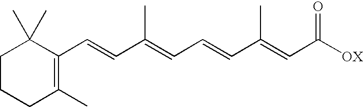

wherein X is a member selected from the group consisting of:

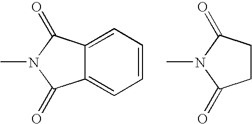

2-cyclohexylethyl; 10-carbomethoxydecyl; 4-hydroxybutyl; cholesteryl; mixed m- and p-vinylbenzyl; and 4-bromobenzyl;

(B) esters of all-trans-retinoic acid having the following formula:

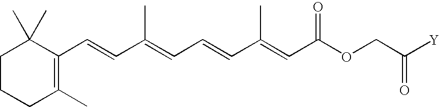

wherein Y is a member selected from the group consisting of: cholesteryloxy; phenyl; 4-bromophenyl; 4-methoxyphenyl; 4-nitrophenyl; 4-hydroxyphenyl; 4-methylphenyl; 4-cyanophenyl; 4-ethoxyphenyl; 4-acetoxyphenyl; 2-naphthyl; 4-biphenyl; 2,5-dimethoxyphenyl; 2,4-dichlorophenyl; 2,4-dimethylphenyl; 3,4-diacetoxyphenyl; 3,4,5-trimethoxyphenyl; and 2,4,6-trimethylphenyl; and

(C) amides of all-trans-retinoic acid having the following formula:

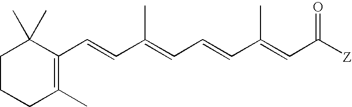

wherein Z is a member selected from the group consisting of: n-propylamino; tert-butylamino; 1,1,3,3-tetramethylbutylamino; 1-morpholino; 4-hydroxyphenylamino; 4-carbomethoxy-2-hydroxyphenylamino; beta-(3,4-dimethoxyphenyl)-ethylamino; 2-benzothiazolylamino; 1-imidazolyl; 1-(2-nicotinoylhydrazolyl); 1-benzotriazolyl; 1-(1,2,4-triazolyl);

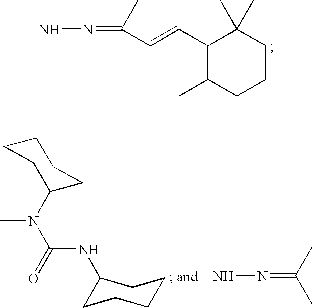

Particularly preferred is all-trans-N-(4-hydroxyphenyl)retinamide, also called fenretinide, which has CAS registry number 65646-68-6, and has the structure:

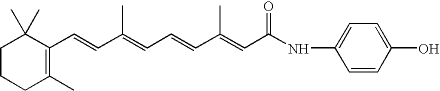

The foregoing compounds can be prepared in accordance with known techniques. See, e.g., U.S. Pat. No. 4,190,594 to Gander et al.; U.S. Pat. No. 4,665,098 to Gibbs.

Additional retinoic acid derivatives that can be used to carry out the present invention include C-Glycoside analogs of N-(4-hydroxyphenyl)retinamide-O-glucuronide. Such compounds and their preparation are known and described in U.S. Pat. Nos. 5,663,377 and 5,599,953, both to Curley et al., the disclosures of which are incorporated by reference herein in their entirety. Such compounds may have the general formula:

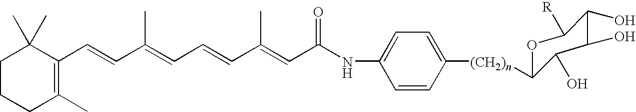

where R is COOH, CH₂OH, or H, and n is 0 or 1.

Specific examples of such compounds include: 4-(retinamido)phenyl-C-glucuronide; 4-(retinamido)phenyl-C-glucoside; 4-(retinamido)phenyl-C-xyloside; 4-(retinamido)benzyl-C-glucuronide; 4-(retinamido)benzyl-C-glucoside; 4-(retinamido)benzyl-C-xyloside; 1-(β-D-glucopyranosyl) retinamide; 1-(D-glucopyranosyluronosyl) retinamide, and bexarotene.

### 2. Microtubule Inhibitor Active Compounds.

Microtubule inhibitors used to carry out the present invention include inhibitors of microtubule structure, stability, and/or function. Such agents include but are not limited to microtubule inhibitors that function via the Vinca tubulin binding domain (e.g., vincristine, vinblastine, vinorelbine, and cryptophycin 52, inhibitors functioning via the Taxane tubulin binding domain (e.g., paclitaxel, docetaxel, and epothilones), and inhibitors functioning via the Colchicine tubulin binding domain (e.g., colchicine, ABT-751, CI-980, and combretastatin). Particular examples are given in Table 1 below.

TABLE 1
Example of compounds that directly or indirectly target microtubule structure.

| | Compound | Structure | Bioactivity | Comments |
|---|---|---|---|---|
| 1 | Docetaxel | 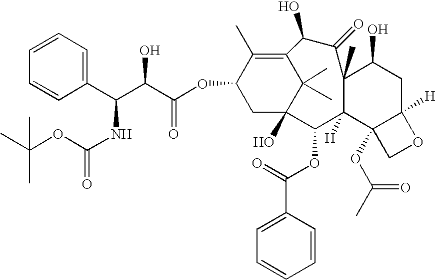 | Microtubule stabilizer | anti-cancer |
| 2 | Taxol | 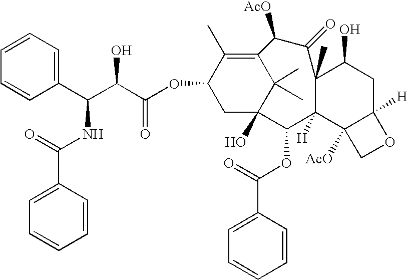 | Microtubule stabilizer, promotes and stabilizes tubulin polymerization | antineoplastic |
| 3 | Podophyllotoxin | 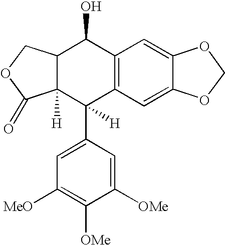 | Tubulin binder, DNA topoisomerase II inhibitor; inhibits microtubule assembly, arrest cell cycle | cytostatic, antineoplastic |
| 4 | Vincristine | 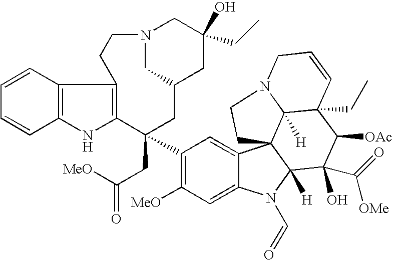 | Microtubule assembly inhibitor; antibiotic | antineoplastic |
| 5 | Vinorelbine | 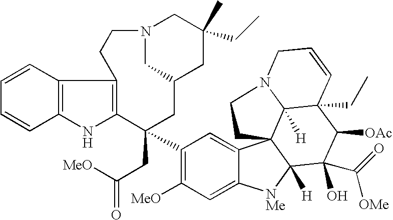 | Microtubule inhibitor | anti-cancer |
| 6 | Griseofulvin | 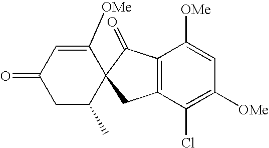 | Interacts with polymerized microtubules and associated proteins; inhibits mitosis in metaphase | antifungal, anti-mitotic |
| 7 | Cytocholasin A | 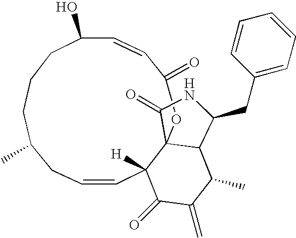 | Microtubule assembly inhibitor | |
| 8 | TN-16 | 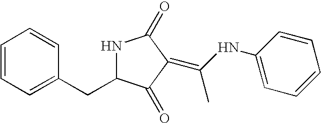 | Microtubule assembly inhibitor | |
| 9 | Myoseverin | 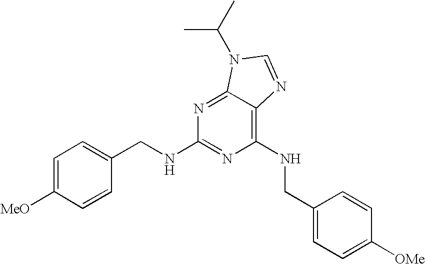 | Microtubule disruptor | |
| 10 | Nocodazole | 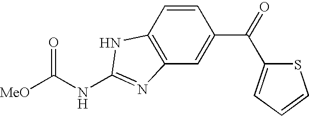 | Microtubule and mitosis inhibitor, inhibits tubulin | |
| 11 | Vindesine | 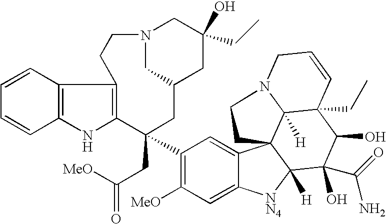 | Microtubule assembly inhibitor; antibiotic | |
| 12 | Phomopsin A | 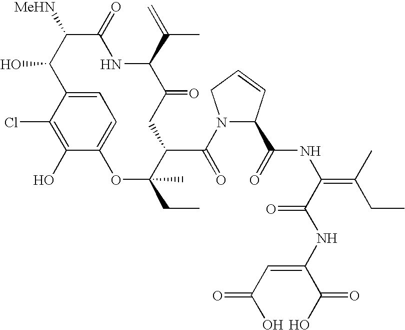 | Microtubule assembly inhibitor | |
| 13 | d-24851 | 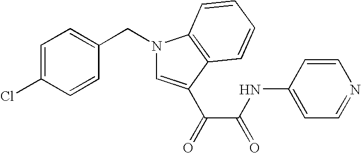 | Microtubule stabilizer | |
| 14 | Monastrol | 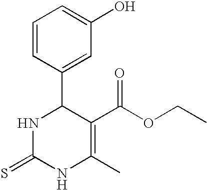 | Mitotic kinesin Eg5 inhibitor(13) | |
| 16 | Adociasulfate-2 | 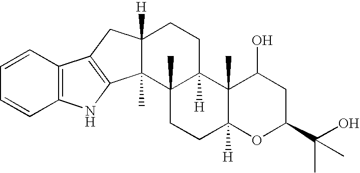 | Inhibitor of kinesin motors (14) | |
| 17 | Terpendole-E | 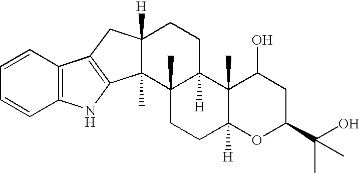 | Arrests cells in metaphase; mitotic kinesin Eg5 inhibitor (15) | |
| 18 | Tubacin | 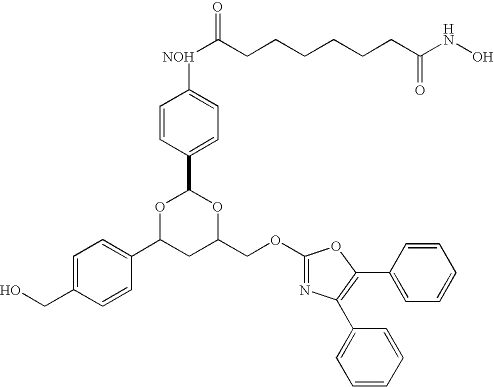 | Microtubule deacetylase (HDAC6) inhibitor (58) | |
| 19 | Scriptaid | 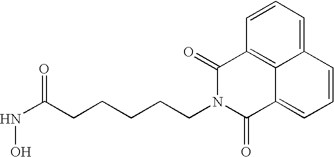 | Deacetylase inhibitor; HDAC inhibitor (26) | |
| 20 | DPD | 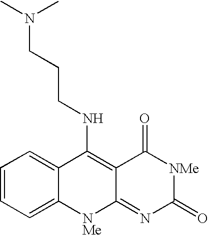 | Inhibits aggresome formation (26) | |
| 21 | C2-8 | 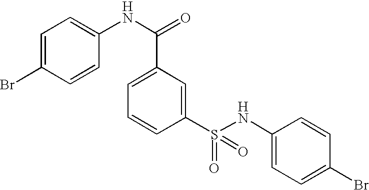 | Poly-Q aggregation inhibitor (38) | |
| 22 | ABT-751 | 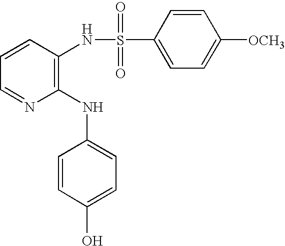 | Binds to Colchicine binding site | |

### 3. Additional Active Compounds and Screening

Additional active compounds can be generated by known techniques, including rational drug design techniques and/or random drug design techniques (or combinatorial chemistry techniques).

In active compounds that interact with a receptor, the interaction takes place at the surface-accessible sites in a stable three-dimensional molecule. By arranging the critical binding site residues in an appropriate conformation, compounds which mimic the essential surface features of the active compound binding region may be designed and synthesized in accordance with known techniques. A molecule which has a surface region with essentially the same molecular topology to the binding surface of the active compound will be able to mimic the interaction of the active compound with its corresponding receptor. Methods for determining the three-dimensional structure of active compounds and producing active analogs thereof are known, and are referred to as rational drug design techniques. See, e.g., U.S. Pat. No. 5,593,853 to Chen; U.S. Pat. Nos. 5,612,895 and 5,331,573 to Balaji et al.; U.S. Pat. No. 4,833,092 to Geysen; U.S. Pat. No. 4,859,765 to Nestor; U.S. Pat. No. 4,853,871 to Pantoliano; and U.S. Pat. No. 4,863,857 to Blalock (the disclosures of all U.S. Patent references cited herein are to be incorporated herein by reference).

In combinatorial chemistry (or random drug design) techniques, large combinatorial libraries of candidate compounds are screened for active compounds therein. Libraries used to carry out the present invention may be produced by any of a variety of split synthesis methods. Split synthesis methods in which a releasable tag is attached to the particle along with the organic compounds of interest are also known as cosynthesis methods. A variety of such methods are known. See, e.g., A. Furka et al., J. Pept. Protein Res. 37, 487 (1991); K. Lam et al., Nature 354, 82 (1991); R. Zuckermann et al., Int. J. pept. Protein Res. 40, 498 (1992); F. Sebestyen et al., Bioorg. Med. Chem. Lett. 3, 413 (1993); K. Lam et al., Bioorg. Med. Chem. Lett. 3, 419 (1993). For example, the library may be a library of organometallic compounds wherein the compound is a metal-ligand complex. The metal in the complex may be an early or late transition metal in high, low or zero oxidation states. The metal may also be any of the main group metals, alkali metals, alkaline earths, lanthanides or actinides. The ligand in the metal-ligand complex may be composed of, or derived from, chiral or achiral forms of cyclopentadienes, amino esters, oxazolidoinones, hydroxy acids, hydroxy esters, hydroxy amides, pyridines, fused pyridines, nitrogen heterocycles, oxazoles, imidazoles, pyrroles, crown ethers, cryptands, carcerands, phosphines, diphosphines, polyphosphines, quinuclidines, quinines, alkaloids, dextrins, cyclodextrins, salens, porpyrins, biaryls, sulfonamides, Schiff bases, metallocenes, monools, diols, polyols, amines, diamines, polyamines, ammonium salts, peptides, proteins, nucleic acids, etc.

As a second example, the library may be a library of non-metal compounds including, but not limited to, chiral or achiral forms of cyclopentadienes, amino esters, oxazolidinones, hydroxy acids, hydroxy esters, hydroxy amides, pyridines, fused pyridines, nitrogen heterocycles, oxazoles, imidazoles, pyrroles, crown ethers, cryptands, carcerands, phosphines, diphosphines, polyphosphines, quinuclidines, quinines, alkaloids, dextrins, cyclodextrins, salens, porphyrins, biaryls, sulfonamides, Schiff bases, metallocenes, monools, diols, polyols, amines, diamines, polyamines, ammonium salts, peptides, proteins, nucleic acids, etc.

The solid supports may be separate from one another, or may be discreet regions on a surface portion of a unitary substrate, which surface portion may be positioned at the interface so that a plurality of the discreet regions are positioned at the interface. Such “chip-type” or “pin-type” solid supports are known. See, e.g., U.S. Pat. No. 5,288,514 to Ellman (pin-based support); U.S. Pat. No. 5,510,270 to Fodor et al. (chip-based support). Separate discreet supports (e.g., particles or beads) are currently preferred. Synthesis of the catalyst library and linking thereof to the discreet solid support may be carried out in accordance with known techniques, such as described in U.S. Pat. No. 5,565,324 (the disclosure of which is incorporated by reference herein in its entirety), or variations thereof that will be apparent to those skilled in the art.

### Formulations and Administration

The active compounds described above may be formulated for administration in a single pharmaceutical carrier or in separate pharmaceutical carriers for the treatment of a variety of conditions. In the manufacture of a pharmaceutical formulation according to the invention, the active compounds including the physiologically acceptable salts thereof, or the acid derivatives of either thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.5% to 95% by weight of the active compound. One or more active compounds may be incorporated in the formulations of the invention, which may be prepared by any of the well-known techniques of pharmacy consisting essentially of admixing the components, optionally including one or more accessory ingredients.

The formulations of the invention include those suitable for oral, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, or intradermal injection. Such preparations may conveniently be prepared by admixing the compound with water or a glycine buffer and rendering the resulting solution sterile and isotonic with the blood.

Formulations suitable for rectal administration are preferably presented as unit dose suppositories. These may be prepared by admixing the active compound with one or more conventional solid carriers, for example, cocoa butter, and then shaping the resulting mixture

Formulations suitable for topical application to the skin preferably take the form of an ointment, cream, lotion, paste, gel, spray, aerosol, or oil. Carriers which may be used include vaseline, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any one active agent, the use of which is in the scope of the present invention, will vary somewhat from compound to compound, patient to patient, and will depend upon factors such as the condition of the patient and the route of delivery. Such dosages can be determined in accordance with routine pharmacological procedures known to those skilled in the art, particularly in light of the disclosure provided herein.

For fenretinide (for systemic treatment), a dose to achieve a plasma level of about 10 uM or 20 uM or 40 uM or 70 uM or greater will be employed, either given orally or intravenously; typically (for oral dosing) 500 or 1000, 2000 or 3000 mg/m² body surface area per day for one week, in every three weeks, or daily is used. Typically, (for intravenous administration) 500 or 1000 or 1500 mg/m²/day, for five days every three weeks, is employed.

For vincristine, a dosage of about 1-2 mg/m² to a maximum dose of 2 mg given intravenously as a single dose, or weekly, or every three weeks, or given as a split weekly dose, or as a continuous infusion over several days, every three weeks, are useful and achievable. For children less that 10 kg, a total dose of 0.05 mg/kg is tolerable given weekly, or every three weeks.

Paclitaxel in castor bean oil (cremophor) intravenous preparations is administered at dosages of 100 or 175 mg/m²/dose, every three weeks or monthly Up to 200 or 270 or 350 mg/m²/day of paclitaxel is employed if administered intravenously as a albumin nanoparticle, every three weeks or four weeks. Other doses and schedules are possible

For ABT-751, typically 100, or 200, or 250 mg/m²/day, or 25 or 100 or 250 mg (fixed dose), is taken orally daily for five days, or for 7 of 21 days, or daily for 21 of 28 days. Other schedules can be employed.

The present invention is explained in greater detail in the following non-limiting examples.

### EXAMPLE 1

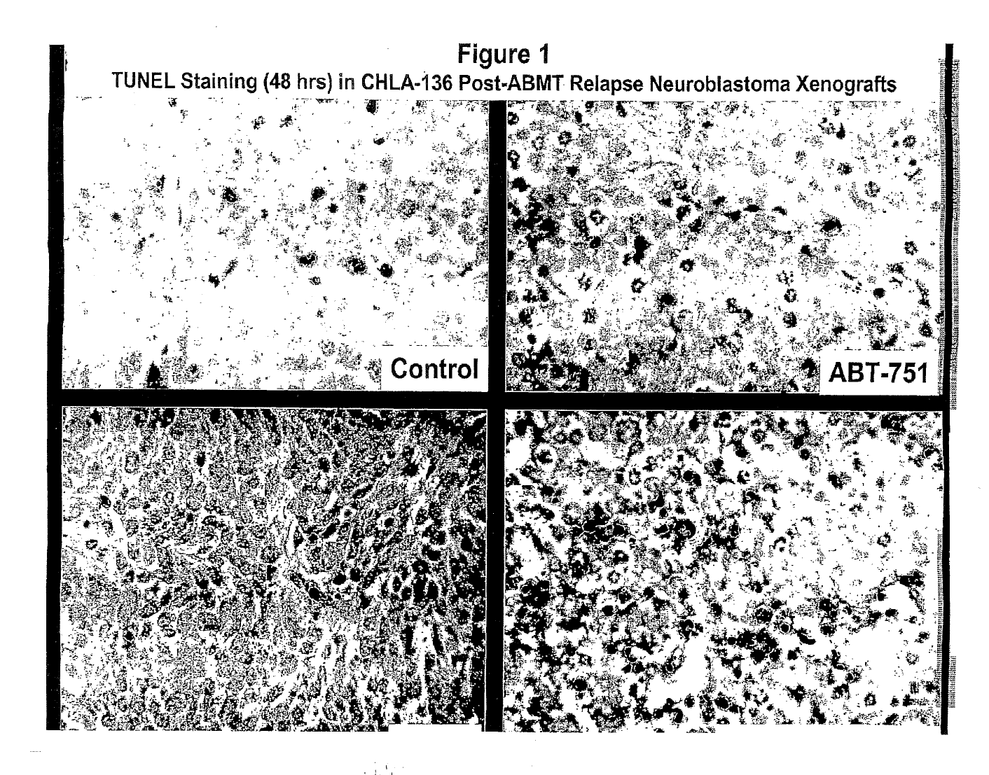

Treating cancer in xenografts with fenretinide+ABT-751. Administration of fenretinide and ABT-751 in combination to nu/nu mice bearing subcutaneous xenografts of multi-drug resistant neuroblastoma xenografts (tumor cell lines are described in: Keshelava N, Zuo J J, Chen P, Waidyaratine S N, Luna M C, Gomer C J, Triche T J, Reynolds C P: Loss of p53 function confers high-level multi-drug resistance in neuroblastoma cell lines. Cancer Research 61:6185-6193, 2001, and xenograft methods are described in: Reynolds, C P, Sun B C, DeClerck Y A, Moats R A: Assessing growth and response to therapy in murine tumor models. Methods in Molecular Medicine Chemosensitivity Vol 2 ed. Blumenthal R D, Totowa: Humana Press pp 335-350, 2005. FIG. 1 shows photomicrographs showing increased apoptotic cell death by TUNEL (detection of internucelosomal DNA breaks using terminal nucleotidyl transferase) assay when the multidrug-resistant human neuroblastoma cell line CHLA-136 was grown as subcutaneous tumor xenografts in immunocompromised murine hosts and were treated with fenretinide+ABT-751 compared to fenretinide or ABT-751 alone.

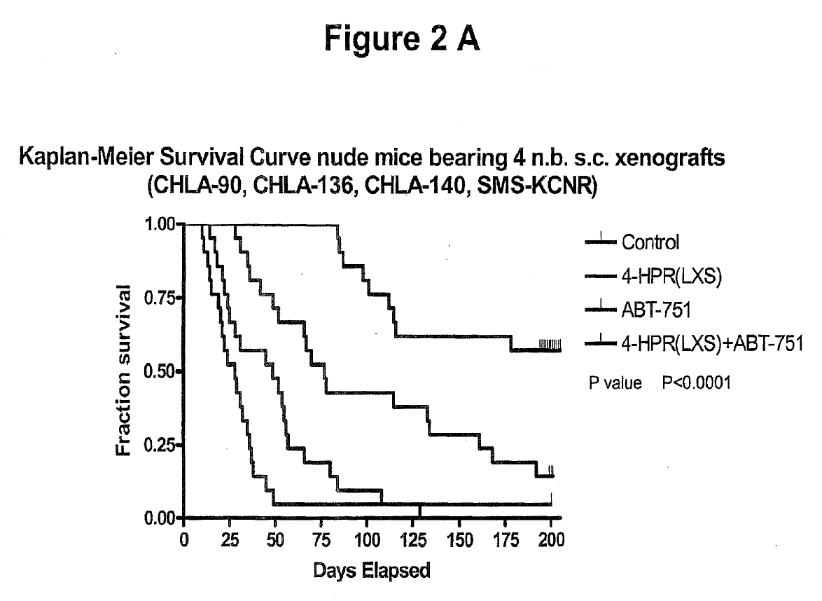

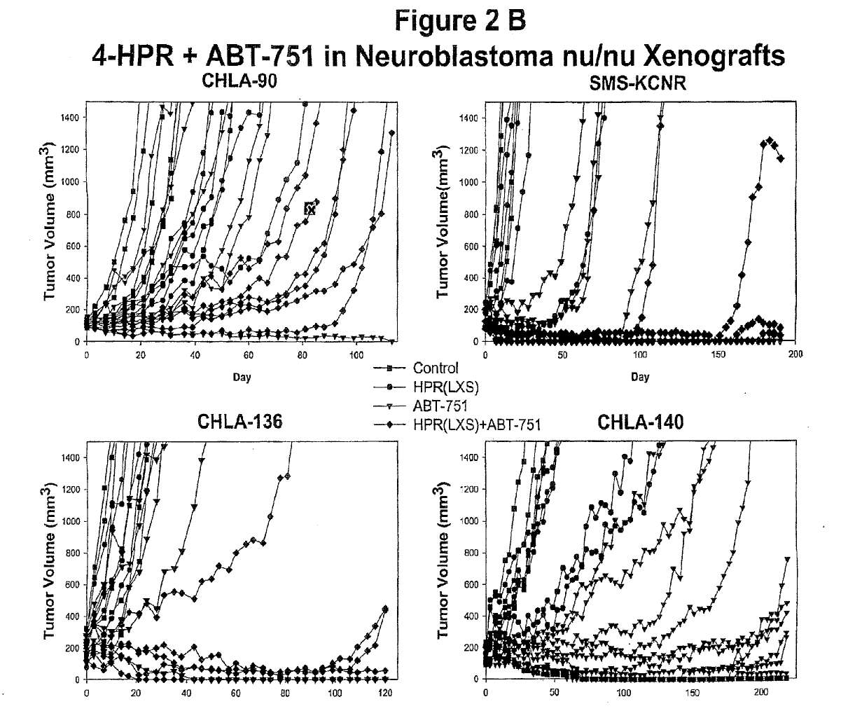

After pilot experiments showed a surprising and striking increase in anticancer activity when fenretinide was combined with ABT-751, we conducted experiments in multiple multi-drug resistant neuroblastoma xenograft models. As shown in FIG. 2 A, these data demonstrated that the event-free survival of immunocompromised mice bearing 4 different human neuroblastoma cell lines (CHLA-90, CHLA-136, SMS-KCNR, and CHLA-140) grown as subcutaneous tumor xenografts was increased by fenretinide+ABT-751 compared to either drug alone even in CHLA-136, a xenograft that was minimally response to fenretinide or ABT-751 as single agents at the drug doses employed. Mice were treated daily with ABT-751, and twice daily with fenretinide for 5 days/week. Fenretinide was given as a powder LXS formulation mixed with water (Maurer B J, Kalous O, Yesair D W, Wu X, Vratilova J, Maldonado V, Khankaldyyan V, Frgala T, Sun B C, McKee R T, Burgess S W, Shaw W A, Reynolds C P. Improved oral delivery of N-(4-hydroxyphenyl)retinamide with novel LYM-X-SORB™ organized lipid complex in mice. Clinical Cancer Research (In Press, 2007)). Both drugs were given to mice by gavage.

FIG. 2 B shows the tumor volume as measured with calipers (twice a week) from the subcutaneous tumors from mice used to derive the event-free survival log-rank analysis shown in FIG. 2 A. Due to achieving sustained complete responses in the SMS-KCNR xenografts, therapy was discontinued about day 60. When multiple recurrences of tumor were observed about day 100, therapy was re-instituted, and anti-cancer activity of the combination was again observed (FIG. 2 B).

### EXAMPLE 2

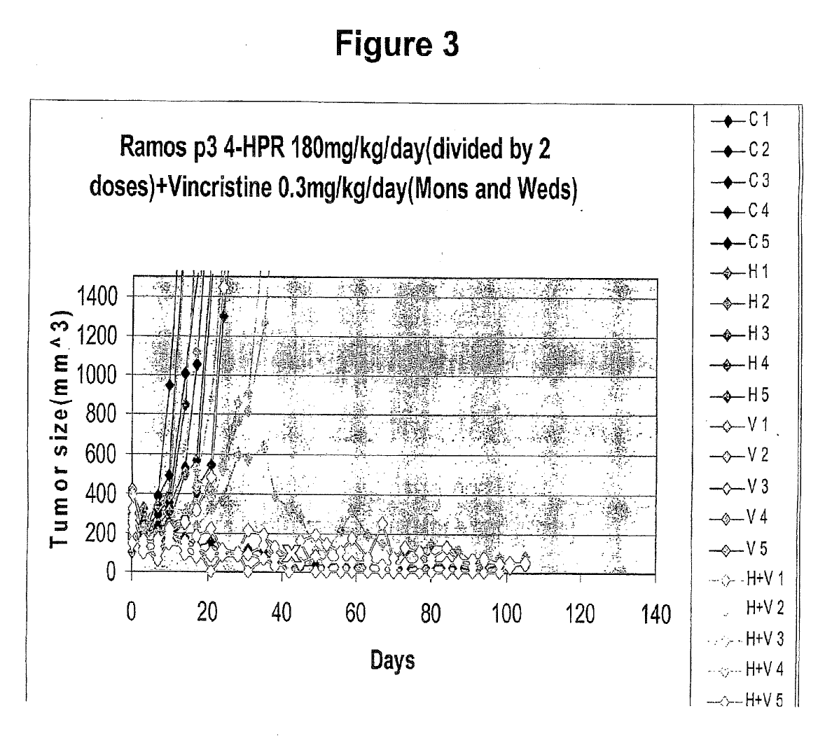

Treating lymphoma xenografts with fenretinide combined with vincristine. FIG. 3 demonstrates that survival of immunocomprised mice bearing the human Ramos Burkitts lymphoma cell line grown as subcutaneous tumor xenografts was increased by fenretinide+vincristine compared to either drug separately even in a tumor cell line minimally responsive to fenretinide or vincristine as single agents at the drug doses employed. Testing of fenretinide+vincristine was carried out as described for fenretinide+ABT-751 in Example 1, except that vincristine was given by i.p. injection twice a week during the 5 day administration of fenretinide.

### EXAMPLE 3

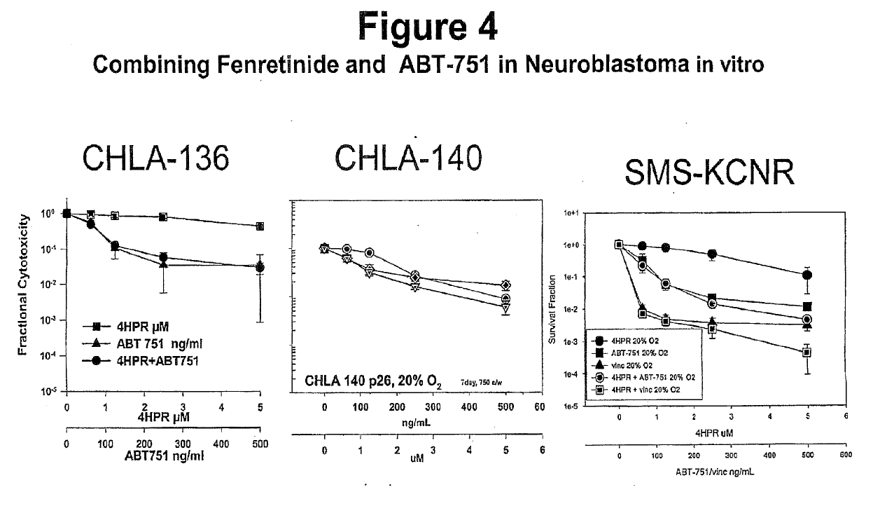

The combination activity of fenretinide+microtubule inhibitors is not readily observed with in vitro assays. The striking anti-cancer activity of combining fenretinide together with microtubule inhibitors was unexpected in light of no known mechanism of action for the drugs would suggest such robust anti-cancer activity would occur with such drug combinations. Moreover, our initial testing of such drug combinations in cell culture failed to demonstrate any drug synergy. FIG. 4 shows data representative of our initial in vitro testing of the microtubule inhibitor ABT-751 in combination with that the in vitro cytotoxicity of the human neuroblastoma cell lines, was not different for fenretinide+ABT-751 compared to either drug alone. Cytotoxicity was determined by DIMSCAN assay (Frgala T, Kalous O, Proffitt R T, Reynolds C P, A fluorescence microplate cytotoxicity assay with a 4-log dynamic range that identifies synergistic drug combinations, Molecular Cancer Therapeutics 2007 March; 6(3):886-97) at 4 days of exposure to various concentrations of the drugs as single agents, or together in a fixed ratio of concentrations. These were the initial data from testing of the combinations. In spite of these data, which did not point toward a synergistic interaction between microtubule inhibitors and fenretinide, we conducted in vivo experiments that obtained the surprising results shown in FIGS. 1 and 2.

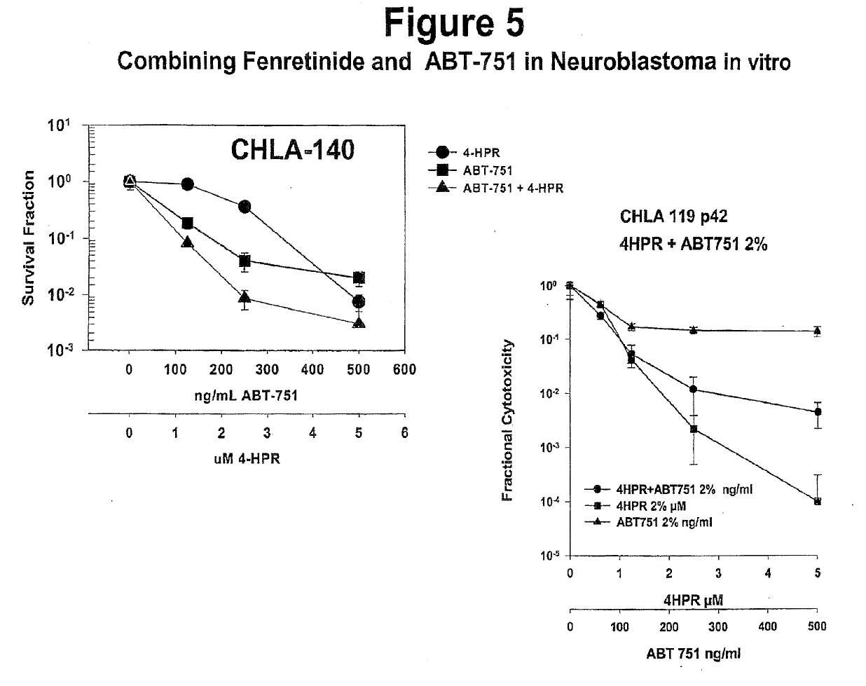

FIG. 5 demonstrates that the in vitro cytotoxicity of fenretinide+ABT-751 for 2 human neuroblastoma cell lines (CHLA-140 and CHLA-119) that were identified after screening a large panel of multi-drug resistant neuroblastoma cell lines. Of all the lines tested to date, only these 2 lines show any increases in cytotoxicity for the combination relative to the single drugs in vitro. Note that unlike the more striking effect seen for all neuroblastoma cell lines tested as xenografts, even in these selected lines, only a modest increase in activity is seen with the combination relative to the single agents. Cytotoxicity was determined. by DIMSCAN assay at 4 days of exposure to various concentrations of the drugs as single agents, or together in a fixed ratio of concentrations.

### EXAMPLE 4

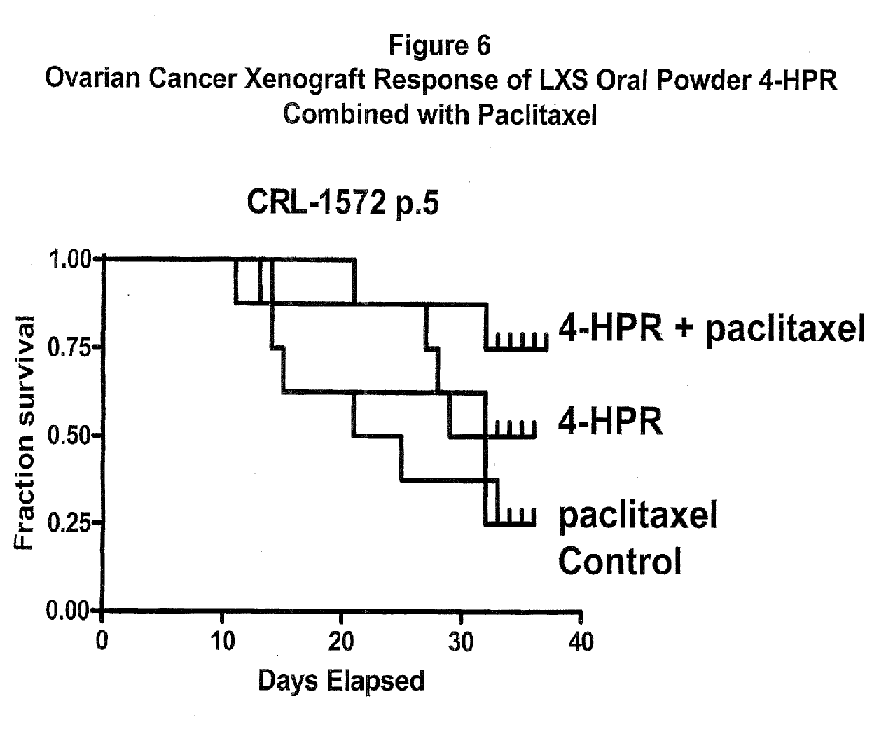

Fenretinide combined with taxanes is active against ovarian cancer Xenografts. Based on the surprising and favorable results observed when combining ABT-751 with fenretinide with neuroblastoma xenografts and vincristine with fenretinide for a lymphoma xenograft, we explored other types of cancer as xenografts and we also sought to test taxanes for a possible increase in activity when combined with fenretinide. FIG. 6 demonstrates that survival of immunocomprised mice bearing human ovarian cancer cell line, CRL-1572, grown as subcutaneous tumor xenografts is prolonged by fenretinide+paclitaxol compared to either drug separately even in a tumor cell line minimally responsive to fenretinide or paclitaxel as single agents at the drug doses employed.

### EXAMPLE 5

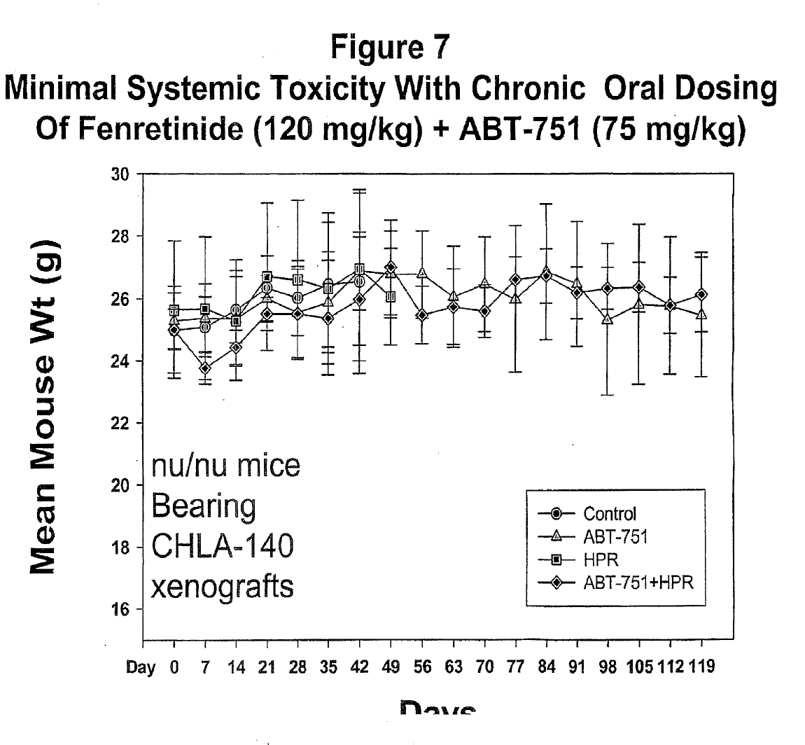

Fenretinide+microtubule inhibitors is well tolerated. In the course of all the mouse xenograft experiments undertaken, we monitored mouse health and the systemic toxicity of the drugs by observation of mouse appearance, activity, and by measuring body weight once a week. All microtubule inhibitors combined with fenretinide were very tolerated, though in some instances, to minimize systemic toxicity, the two drugs in combination were given at doses less than could be obtained for each drug alone. FIG. 7 illustrates that the combination of ABT-751+fenretinide is well tolerated in mice as demonstrated by the lack of weight loss from the mice.

The foregoing is illustrative of the present invention, and is not to be construed as limiting thereof. The invention is defined by the following claims, with equivalents of the claims to be included therein.

1. A method of treating a hyperproliferative disorder in a subject in need of such treatment, comprising administering to said subject, in combination, a treatment effective amount of:
(a) a retinoid or a pharmaceutically acceptable salt thereof, and
(b) a microtubule inhibitor.

2. A method according to claim 1, wherein said retinoid is fenretinide.

3. A method according to claim 1, wherein said a microtubule inhibitor is a Vinca alkaloid binding domain inhibitor.

4. A method according to claim 3, wherein said microtubule inhibitor is vincristine.

5. A method according to claim 1, wherein said a microtubule inhibitor is a Colchicine binding domain inhibitor.

6. A method according to claim 5, wherein said a microtubule inhibitor is ABT-751.

7. A method according to claim 1, wherein said a microtubule inhibitor binds to the taxane binding domain.

8. A method according to claim 7, wherein said a microtubule inhibitor is paclitaxel or docetaxel.

9. The method of claim 1, wherein said subject is a mammalian subject.

10. The method of claim 1, wherein said subject is a human subject.

11. The method of claim 1, wherein said hyperproliferative disorder is cancer.

12. The use of a retinoid or pharmaceutically acceptable salt thereof for the preparation of a medicament for the treatment of a hyperproliferative disorder in combination with a microtubule inhibitor in a subject in need thereof.

13. The use of claim 12, wherein said retinoid is fenretinide or a pharmaceutically acceptable salt thereof.

14. (canceled)

15. The pharmaceutical formulation of claim 21, wherein said a microtubule inhibitor is a Vinca alkaloid binding domain inhibitor.

16. The pharmaceutical formulation of claim 21, wherein said microtubule inhibitor is vincristine.

17. The pharmaceutical formulation of claim 21, wherein said a microtubule inhibitor is a Colchicine binding domain inhibitor.

18. The pharmaceutical formulation of claim 21, wherein said a microtubule inhibitor is ABT-751.

19. (canceled)

20. The pharmaceutical formulation of claim 21, wherein said a microtubule inhibitor is paclitaxel or docetaxel.

21. A pharmaceutical formulation comprising:
(a) a microtubule inhibitor;
(b) a retinoid or pharmaceutically acceptable salt thereof; and
(c) a pharmaceutically acceptable carrier,
wherein the microtubule inhibitor and the retinoid or pharmaceutically acceptable salt thereof are present in an amount effective for the treatment of a hyperproliferative disorder.
